(19) United States
(12) Patent Application Publication
(10) Pub. No.: US 2024/0368517 A1
(43) Pub. Date: Nov. 7, 2024

(54) CELL CULTURE DEVICE AND MOVEMENT SYSTEM

(71) Applicants: UNIVERSITETET I OSLO, Oslo (NO); OSLO UNIVERSITETSSYKEHUS HF, Oslo (NO)

(72) Inventors: Stefan Johannes Karl KRAUSS, Oslo (NO); Mathias BUSEK, Oslo (NO); Mikel Amirola MARTINEZ, Oslo (NO); Aleksandra AIZENSHTADT, Oslo (NO)

(21) Appl. No.: 18/580,711

(22) PCT Filed: Jul. 19, 2022
(86) PCT No.: PCT/EP2022/070215

§ 371 Date: Jan. 19, 2024

(30) Foreign Application Priority Data
Jul. 19, 2021 (GB) 2110366.8
Jul. 1, 2022 (GB) 2209729.9

(51) Int. Cl. C12M 3/06; C12M 1/00; C12M 1/04

(52) U.S. Cl. CPC C12M 27/14; C12M 23/24; C12M 29/04; C12M 29/10

(57) ABSTRACT

A cell culture apparatus including a cell culture device and a movement system for moving the cell culture device. The cell culture device includes at least two reservoirs for holding a liquid cell medium, one or more chambers for culturing of living cells, tissues or living organoids and at least two perfusion channels connecting the reservoirs. The chambers are separated from at least one of the perfusion channels by a semipermeable barrier for selective transport of cell media and/or for selective growth or migration of living cells. The movement system is configured to move the cell culture device with a movement axis in a tilted orientation in order to move the lowest point of a flow loop through all points within the flow path of the flow loop and thereby generate gravity driven circulation of the liquid cell medium around the flow loop.

The present invention relates to a cell culture device and in particular to a cell culture device using a directional flow of a liquid cell medium. Related methods are also described herein.

Complex in vitro models are needed to recapitulate higher-level anatomical and physiological or pathological aspects of human or animal biology. Organ-on-a-chip (OoC) technology is quickly advancing as a platform for such complex in vitro models. OoC technology represent aspects of human organs and tissues and promises to reproduce human physiology in a way that resembles the human situation good enough for predictive testing of interventions. This is key for preclinical testing of novel drugs and for personalization of drug testing. Clear evidence shows that OoC technology is on the verge of widespread impact on academia and the pharmaceutical industry as much-needed physiological models and potential alternatives to animal testing. For reliable market penetration, simple, versatile and scalable platforms are needed.

Current OoC systems that are optimized for standard cell culture labs rely on pumps, 2D gravity driven perfusion or gravity driven tilting platforms for bi-directional liquid flow but mostly lack important features like a directed flow, and control of differential cultivation parameters like oxygen levels. In common systems for directed flow, connection to pumps for liquid exchange are complicated, adsorb chemical reagents and are difficult to scale, and standard processes like cell loading or media exchange are complicated. This limits the reproducibility and scalability of directional flow OoC technology. A further limitation of many, especially academic, OoC systems with directional flow—and microfluidics in general—is its high entry barrier for cell biologists to work with them. Especially the pharmaceutical industry is in need of a robust and scalable directed flow platform that allows high numbers of parallel experiments.

Viewed from a first aspect, the present invention provides a cell culture apparatus comprising: a cell culture device; and a movement system for moving the cell culture device;
the cell culture device comprising: at least two reservoirs for holding a liquid cell medium, one or more chambers for culturing of living cells, tissues or living organoids and at least two perfusion channels connecting the reservoirs; wherein the one or more chambers are separated from at least one of the perfusion channels by a semipermeable barrier for selective transport of cell media and/or for selective growth or migration of living cells; and wherein the reservoirs and perfusion channels are in fluid communication with one another in order to form a flow loop having a flow path for a one-way gravity-driven flow of the liquid cell medium when the cell culture device is tilted and moved by the movement system;
a movement axis being defined as an axis of the cell culture device that passes through the cell culture device with the flow loop being located around the movement axis; and
the movement system being configured to move the cell culture device with the movement axis in a tilted orientation in order to move the lowest point of the flow loop through all points within the flow path of the flow loop and thereby generate gravity driven circulation of the liquid cell medium around the flow loop, wherein the tilted orientation places the movement axis at an angle in the range 5° to 85° away from the vertical.

With the apparatus of the first aspect it is possible to generate a true directional circulating movement of liquids, giving clear advantages over the bi-directional tilting arrangements of the prior art. The low point, i.e. the point to which gravity driven flow moves, is circulated around all points of the flow path of the flow loop, rather than simply being moved from side to side as in bi-directional systems. This gives significant benefits in relation to applications requiring modelling or simulation of microphysiological systems. Advantageously there are no pumps for movement of the liquid cell medium in the flow loop. Whilst platforms with pumps can create a better motion of fluid than bi-directional tilting these pumped systems have other limitations compared to the proposed cell culture device, such complexity and expense giving a high entry barrier for end users, the need for specific ancillary equipment, an absence of recirculating fluidic flow, bubble formation issues and so on. Since the movement involves tilting the flow speed can be controlled by tilting angle and rotational speed, e.g. to fit with typical physiological ranges as discussed below.

By using this type of system it is possible to transport the liquid cell medium, which may comprise cell cultivation media or other liquids, in a one-way (directional) path repeatedly, circulating the liquid (e.g. blood and cells) directionally in a ring of fluidic channels. This allows the formation of directional perfused endothelialized blood channels including arterial and venous components, circulating immune cells and serial coupling of organs without the need of external pumps. The proposed cell culture device is simple and scalable while it offers a significantly broadened functionality.

The movement in a tilted orientation is a movement of the device whilst the axis is at an angle to the vertical axis. This angle is an angle in the range of 5° to 85° away from the vertical axis, with the flow of the liquid cell medium hence being flow downward along a sloping path that may be angled in the range 5° to 85° to the horizontal. Optionally the working range for the angles is 20° to 80°, or more narrowly 50° to 80° away from the vertical, i.e. in the range 10° to 70° or more narrowly 10° to 40° to the horizontal axis. In one example the movement axis is tilted at 20° from the horizontal. The movement in a tilted orientation can be any movement that promotes circulating gravity driven flow about the flow loop, and advantageously is a continuous movement providing for a continuously moving flow. Alternatively the movement system may be configured to provide a movement involving pauses at desired points, i.e. a move-stop-move-stop sequence, or a movement of varying speed and/or a movement for creating a varying flow pressure. This can better simulate regular pulsatile flows as found in real physiological systems or irregular pulsatile flows such as present in arrhythmic conditions. The movement system may comprise a controller that can be programmed with a range of movement patterns. In example embodiments the cell culture device is configured so that the flow characteristics, e.g. one or more of speed, shear, repetition rate fit within known ranges for physiological flow such that the cell culture device can more accurately simulate physiological processes within a living organism.

The cell culture device includes at least one chamber for culturing of living cells, living tissues or living organoids, such as for simulating physiological processes and hence allowing for “Organ-on-a-Chip” (OoC) capabilities. The living cells may for example comprise body tissues and/or pathogens like bacteria, viruses, fungi or other cell types. The chamber is separated from one of the perfusion channels by said semipermeable barrier, which is for selective transport of cell media and/or for selective growth or migration of living cells, such as by allowing cell nutrients and cell waste to pass but preventing at least some types of cells from passing. Some types of cell may be allowed to pass, including for example selected pathogens like bacteria, viruses, fungi, e.g. to simulate circulation of pathogens within the body. Thus, the barrier advantageously acts as a semi-permeable and/or selectively permeable barrier between the perfusion channel and the chamber, but also allows for passage of certain cell media and hence provides a way of connecting the liquid cell medium to the at least one chamber. This barrier may be of a known type for use in OoC devices and similar in vitro systems, such as an extra cellular matrix (ECM) barrier. The barrier can be selected according to the desired application for the cell culture device.

An example barrier comprises a biomaterial or extracellular natural material selected and placed to achieve the desired barrier effect, such as for allowing cell nutrients and cell waste to pass but preventing at least some types of cells from passing. The barrier may allow vascularization, immune cell migration and other cell penetration such as tumour cell invasion. Alternatively or additionally the barrier may enable innervation. The barrier may comprise extracellular matrix hydrogel, for example Matrigel®, or/and a synthetic hydrogel, for example PEG. Alternatively the barrier may comprise a polymer membrane with defined pore size e.g. made of PC, PET or PMMA.

In some example embodiments there are two barriers at two locations about the chamber, at points where the chamber joins with first and second perfusion channels of the at least two perfusion channels. Thus, the two barriers may provide for transport of certain media between the chamber and each of both of the two perfusion channels. This may be done, for example, at two sides of the chamber, such as at two opposite sides thereof. There may be multiple chambers on the cell culture device, each having one or more connections to the perfusion channels by respective barriers. Thus, the cell culture device can facilitate in vitro modelling of processes using multiple separate stages, such as combining both arterial and venous blood perfusion and allowing formation of perfusable microvasculature or vascularized living tissue or organoids between them. In another application the system may allow lymph drainage or bile duct drainage. Moreover, this configuration can be used to model the digestive or the urinal tract with the second circuit forming transporting the nutrients or urine.

A part of the flow loop may be configured to allow contact of the liquid cell medium with a gas from outside of the flow loop, such as atmosphere or a gas in a gas circulation system. This may allow for additional processes such as exchange of oxygen between the gas and the liquid cell medium. Thus, in some examples a part of the flow loop may be configured for contact of the liquid cell medium with an oxygen-containing gas or an oxygen depleting gas. It will be appreciated that the exchange of gases will depend on the relevant partial pressures, for example contact with air can be used to introduce oxygen to low oxygen liquids. Nitrogen gas or mixtures of nitrogen, carbon dioxide and air may be used to remove oxygen. A contact of the liquid cell medium with a gas from outside of the flow loop may be achieved by an opening that exposes the liquid cell medium within the flow loop to atmosphere, i.e. to air, or alternatively to some other gas source. If air or some other oxygen containing gas is allowed to contact the liquid cell medium then this may form an asymmetric oxygen content in the liquid cell medium. This can be useful in simulation of physiological processes such as arterial and venous blood flow. The part of the flow loop that allows for contact with a gas may for example be one of the reservoirs or one of the of the perfusion channels.

The movement axis for the cell culture device may be conveniently defined as an axis normal to a plane of the device. During movement of the device by the movement system the movement axis may be tilted in the sense that it is angled to the vertical thereby positioning lifting one section of the flow loop higher than another section of the flow loop. The position of the tilted axis may move in order to change the section(s) of the flow loop that are higher and lower relative to one another.

The flow loop involves flow in a circuit, i.e. starting and ending at the same point, and thus necessarily extends generally about a loop that can be considered to define a planar reference surface, which may hence be seen as a reference plane of the cell culture device. It will be appreciated that the flow loop may involve a circulation of flow of the liquid cell medium in such a reference plane and around the movement axis.

The flow loop provides the fluid path for circulation of fluid in a loop about the movement axis, and this circulation of fluid is driven by gravity. If the movement axis were to be kept immobile then the gravity driven flow will not occur. In example embodiments if the movement axis is vertical then the reference plane is horizontal and there would be no circulation of fluid, although it may gather in points of the loop with a lower depth in the plane, e.g. such as for reservoirs that extend deeper than other parts.

In some examples, the tilted movement axis may be moved in a rotational fashion so that a reference point on the movement axis traces around the circumference of a two dimensional shape, such as a circle, oval, stadium or rectangle. The reference point can be any point spaced apart from the intersection of the movement axis with the cell culture device. This may be an intersection of the movement axis with the reference plane of the cell culture device.

Alternatively or additionally the movement in a tilted orientation may include a rotation of the device about the movement axis, e.g. such that a reference point on the flow loop moves in a circle about the tilted movement axis.

The rotation of the movement axis and/or the rotation of the device about the movement axis may be at a speed that is selected to facilitate modelling of the physiological process for which the cell culture device is to be used. The movement system may advantageously be configured to allow for varying flow speeds and/or to create varying flow pressure. In some examples the rotation, and hence the circulation of the liquid cell medium about the flow loop, is done at a speed of between 0.1 to 100 cycles/revolutions per minute, for example about 1 to 20 revolutions per minute. One example system, which has been found to give good results, operates at seven revolutions per minute.

The flow loop may be fully planar in context of the reference plane, i.e. with a lowest point that is always on the plane for each part of the loop. This is a simple arrangement with advantages for ease of manufacture and straightforward calculations in relation to modelling of the flow. However, more complex flow paths can also provide advantages and the flow loop may hence include a lowest point that varies in depth relative to the reference place, e.g. to provide deeper wells for liquid at certain points, to increase or decrease the angle of flow and provide varying flow characteristics at different parts, and/or to promote one-way flow, such as by means of ‘steps’ in the flow path that act to restrict reverse flow. Such steps may for example comprise fluid paths with inlet and outlet at differing levels relative to the reference plane.

As noted above the system may be configured for continuous flow or a varying speed of flow, including a stop-start speed, and/or for varying pressure. This can be achieved by appropriate control of the tilting movement, such as by introducing pauses in the movement pattern and/or changing the tilting angle.

The restriction of reverse flow is a potentially beneficial feature no matter how it is achieved, and the flow path may hence include one or more suitable flow reversal restriction feature(s), which may comprise any feature for preventing or restraining reverse flow. In this context reverse flow is a flow along the loop in a direction going the opposite way to the flow direction in the intended normal use of the cell culture device. The feature(s) for preventing reverse flow may include one or more of: changes in depth relative to the reference plane, as discussed above; capillary stop valves; and/or surface treatments promoting decreased wetting, e.g. directionally decreased wetting. Localised or global surface treatments affecting wetting may involve mechanical features such as features manufactured by engraving, e.g. laser engraving or chemical/physical induced increasing or reducing of hydrophilicity e.g. by applying a UV/ozone or plasma treatment possibly combined with a masking technology for local changes. Alternatively or additionally they may comprise coatings or surface modifying treatments. Decreasing wetting will promote formation of a meniscus that will restrain reverse flow. Decreasing the wetting will increase the contact angle between the liquid and the surface of the reservoir, thereby restraining reverse flow. A capillary stop valve may use a restricted dimension of the flow path and/or a shape of the flow path to restrain reverse flow. This also involves surface tension and thus can be advantageously combined with a surface treatments to increase the wettability thus restraining the liquid in the channels.

The flow loop may comprise a looped flow path that passes, in sequence, from a first reservoir, then through a first perfusion channel and past a first barrier at a first side of the chamber, then to a second reservoir, then from the second reservoir, through a second perfusion channel and past a second barrier at a second side of the chamber, then returning to the first reservoir. Thus, the movement with tilting that generates the gravity driven flow may move the lowest point of the flow loop in sequence through flow path in the same sequence. This should produce a one-way flow around the flow loop. As noted above other features may be present, such as at inlets and outlets of the reservoirs, to restrict reverse flow.

The reservoirs may each comprise a volume for receiving liquid cell medium and optionally for contact of the liquid with an external agent, such as a gas as discussed above. The dimensions of the reservoirs may include: width in the range 2 to 5 mm, e.g. about 3 mm; length in the range 10 to 30 mm, e.g. about 15 mm or about 20 mm; and depth in the range 2 to 6 mm, e.g. about 4 mm. The volume of the reservoirs may be 40 to 900 μL, optionally 60 to 500 μL.

The perfusion channels may each extend from the outlet of one reservoir to the inlet of another reservoir, passing by at least one chamber, e.g. with a barrier providing a link to the chamber at a mid-portion of the channel. The perfusion channels may have a cross section in the range 0.01 mm² to 4 mm², optionally 0.1 mm² to 1 mm², such as a round or rectangular cross-section with largest dimensions from 0.1 mm to 2 mm, optionally 0.5 mm to 1.5 mm. The length of each perfusion channel may be in the range 10 to 30 mm.

The chamber may comprise a volume for receiving the living cells or living organoids, which may be contained within a suitable liquid medium. This may be the same liquid cell medium as that in the flow loop. The chamber may be sealed apart from the contact with the perfusion channels via the barrier(s). The dimensions of the chamber may include: width in the range 0.5 to 2 mm, e.g. about 1 mm; length in the range 2 to 30 mm, e.g. about 6 mm or about 15 mm; and depth in the range 0.5 to 2 mm, e.g. about 0.75 mm or about 1 mm. The volume of the chamber may be 2.5 to 120 μL, optionally 10 to 60 μL. If multiple chambers are present then they may have similar or different sizes as well as being for similar or different cells or organoids.

To separate the cells/organ models from direct flow and shear forces, a separation layer may be introduced between the cell chamber(s) and the perfusion channels. This separation layer may consist of extracellular matrix materials (ECM) possibly mixed with one or more cells. To prevent the ECM from entering the perfusion channels, surface tension effects at the air-liquid interface are utilized. A convex meniscus forms when the channel cross section is changed abruptly. A possible structure is a localized reduction of the channel height between the perfusion channel(s) and the cell chamber(s). To allow cell outgrowth in the ECM, the cross section of the channel between the perfusion channel(s) and the cell chamber(s) is reduced to a range of 0.1 to 0.4 mm. The channel between the perfusion channel(s) and the cell chamber(s) may be reduced in height by increasing the thickness of the upper layer in order to reduce the height of the ceiling. The reduced channel covers the whole length of the culture chamber and may have a height of between 0.2 to 1 mm wide.

Alternatively perfusion channel(s) and cell chamber(s) may be also separated horizontally with the same approach by locally reducing the contact area between them such that a meniscus is formed on-top of the cell chamber and the liquid will flow above the culture chamber. In this configuration, the perfusion channels and the cell chambers may be of the same width (1 to 10 mm) but compromise different heights (0.2 to 1 mm) and lengths (perfusion channel as mentioned before and cell chamber between 1 to 10 mm). The separation layer would contain a “window” with a smaller area e.g. 0.1 to 0.5 mm less width and length as the cell chamber size and would be between 0.2 to 1 mm high. The main advantage of this configuration is that it provides a much higher contact area between perfusion channel and the culture chamber.

The cell culture device may include the liquid cell medium therein and/or the chamber(s) may include therein a living cell or living organoid. When the cell culture device includes the liquid cell medium there may be a volume of 50 to 600 μL, optionally 200 to 400 μL of the liquid cell medium.

The dimensions of the cell culture device as well as the tilting speed and angle may be configured to provide flow rates within the range of physiological flow rates. For example, they may be configured to provide a flow rate of 15 to 80 μL/s, optionally 20 to 60 μL/s and in some examples 23 to 47 μL/s. The mean flow rate may lie within these ranges. Optionally the peak flow rate may also lie within at least the larger ranges, e.g. 15 to 80 μL/s. This is, for example, consistent with flow rates within smaller arteries or bigger arterioles. It will be appreciated that the cell culture system can be tuned to allow for other flow rates as desired by varying the dimensions as well as controlling the tilt angle and speed of movement (e.g. speed of rotation).

The dimensions of the cell culture device as well as the tilting speed and angle may be configured to provide wall shear stress (WSS) within the range of physiological shear stresses. For example, they may be configured to provide a WSS in the range of 0.1 to 100 dyne/cm², optionally 0.1 to 10 dyne/cm², which is consistent with WSS within veins, or 10 to 100 dyne/cm², which better replicates WSS in arteries. The mean WSS may lie within these ranges. Optionally the peak WSS may also lie within at least the larger ranges, e.g. 0.1 to 100 dyne/cm². With flow rates in the range 23 to 47 μL/s and with a liquid cell medium having viscosity 1 mPas then the WSS will be in the range for veins. It will be appreciated that the cell culture system can be tuned to allow for other WSS as desired by varying the dimensions as well as controlling the tilt angle and speed of movement (e.g. speed of rotation).

The tilting angle along both axes is φ and θ can be expressed with the maximum tilt α, the rotation frequency f and time t:

$$φ ( t ) = α * sin ( 2 π * f * t )$$

$$θ ( t ) = α * cos ( 2 π * f * t )$$

The tilt leads to a height difference Δh along the axis that produces a gravity-induced pressure difference Δp (gravity constant g=9.81 m/s², density ρ=1000 kg/m³) (i.e. the vertical axis):

$$Δ p = Δ h * g * ρ$$

This pressure is countered by the wall friction of the perfusion channels:

$$Δ p = \frac{8 * μ * l * Q}{π * {r}_{h}^{4}} = {R}_{h} * Q$$

Whereby, the viscosity μ is 1 mPa*s, l is the channel length and rh is the hydraulic radius of the channel can be summarized to the hydraulic resistance Rh. The gravity-induced pressure difference leads to a flow Q(t) that depends on the height difference Δh that not only depends on the tilt but also the liquid levels in the source (h₁) and sink reservoir (h₂):

$$Δ h = l * sin ( φ ( t ) ) + {h}_{1} - {h}_{2}$$

In turn, the liquid levels can be expressed by the volume change (flow rate) in the reservoirs divided by the surface area of the reservoirs (A) leading to following differential equation:

$$Q ( t ) = \frac{g * ρ}{{R}_{h}} [ ( l * sin ( α * sin ( 2 π f * t ) ) ) + \frac{\int Q d t}{A} ]$$

For infinite reservoir volumes, the general solution will be approximately:

$$Q ( t ) \approx \frac{g * ρ}{{R}_{h}} * l * cos ( 2 π f * t )$$

This flow would be bidirectional and only depending on the platform configuration and the fluidic resistance. Nevertheless, in the 3d-tilting configuration three additional effects needs to be taken into consideration: (1) the liquid in the reservoirs is tilted by θ(t) and (2) the reservoir volume is finite and reservoirs can run dry and (3) a capillary-stop effect occurs at the air-liquid interfaces in the reservoirs.

(1) Will lead to different liquid levels (h₁, h₂) at the in/and outlet. Together with (2) it will cause the flow rate to be zero when the liquid level at the outlet is lower than the tilt of the reservoir (lᵣₑₛ*sind(θ(t)). Once a reservoir runs dry, an air-liquid interface forms at the reservoir outlet and the capillary-pressure needs to be overcome to induce a negative flow (from the inlet of the receiving reservoir back to the outlet of the source reservoir). The capillary force pc can be calculated as follows:

$${p}_{c} = 2 σ * cos δ / {r}_{c}$$

With σ denoting the surface tension of the liquid (for cell culture media the surface tension may be between 40 and 70 mN/m), δ marking the wetting angle of the air-liquid interface (the wetting angle may be between 1° and 30° after surface treatment) and rc representing the hydraulic radius of the reservoir outlet. The capillary stop valve acts as a diode as the gravity-induced pressure Δp needs to be higher than pc to induce a liquid exchange, and as the liquid in the receiving reservoir is tilted by θ the respective height difference is not high enough to overcome the capillary pressure.

The flow rate Q will result in a WSS for adherent cells at the perfusion channel walls. At the bottom and top of the perfusion channels it is (for the case in which the perfusion channels may be approximated to comprise a rectangular cross section):

$${τ}_{cells} = \frac{6 * μ * Q}{{z}^{2} * w}$$

Where μ is the earlier mentioned viscosity, z is the perfusion channel height and w is the perfusion channel width.

Taken together, the WSS can be optimised based on the cross section of the flow path(s) and the reservoirs, localized methods to change the wetting, the design of the reservoir in/outlets and can readily be controlled via a controllable tilting speed ω and angle α, the amount of liquid filled in the reservoirs V₀ and the liquid filled in the system.

The cell culture device is preferably used as an organ-on-a-chip device and thus may contain a combination of a liquid cell medium, barriers, and living cell or living organoids that are known for organ-on-a-chip devices.

Viewed from a second aspect, broader than the first aspect, the present invention provides a cell culture device as described above, separate to the cell culture apparatus and its movement system. That is to say, it is considered that the cell culture device is novel and inventive in its own right. Moreover, it is considered that this applies even without a restricted range of angles for the tiling of the device. Thus, in such an aspect, the invention provides a cell culture device comprising: at least two reservoirs for holding a liquid cell medium, one or more chambers for culturing of living cells or living organoids and at least two perfusion channels connecting the reservoirs; wherein the one or more chambers are separated from at least one of the perfusion channels by a semipermeable barrier for selective transport of cell media and/or for selective growth or migration of living cells; and wherein the reservoirs and perfusion channels are in fluid communication with one another in order to form a flow loop having a flow path for a one-way gravity-driven flow of the liquid cell medium when the cell culture device is tilted and moved by the movement system; wherein a movement axis is defined as an axis of the cell culture device that passes through the cell culture device with the flow loop being located around the movement axis; and wherein the cell culture device is configured such that when it is moved with the movement axis in a tilted orientation in order to move the lowest point of the flow loop through all points within the flow path of the flow loop then this will generate gravity driven circulation of the liquid cell medium around the flow loop.

Optionally, as discussed above, the cell culture device can be used within a cell culture apparatus that also includes a movement system for providing the required movement with reference to the movement axis. Also, or alternatively, the cell culture device may be configured for a movement in which the tilted orientation places the movement axis at an angle in the range 5° to 85° away from the vertical, or narrower ranges of angle as is described above as an optional feature of the first aspect. The cell culture device of this second aspect may also or alternatively include any of the other optional features discussed above in relation to the first aspect.

Viewed from a third aspect, the present invention comprises a cell culture apparatus comprising: a cell culture device; and a movement system for moving the cell culture device;
the cell culture device comprising:
a first flow loop comprising a first reservoir for holding a liquid cell medium and a first perfusion channel, wherein the first reservoir comprises a first reservoir first end and a first reservoir second end, and wherein the first perfusion channel provides fluid communication between the first reservoir first and the first reservoir second end thereby providing a looped flow path for a one-way gravity-driven flow of the liquid cell medium within the first flow loop when the cell culture device is tilted and moved by the movement system, and
one or more chambers for culturing of living cells or living organoids; wherein
the one or more chambers are separated from the first perfusion channel by a semipermeable barrier for selective transport of cell media and/or for selective growth or migration of living cells;
wherein a movement axis is defined as an axis of the cell culture device that passes through the cell culture device;
and wherein the cell culture device is configured such that when the cell culture device is moved with the movement axis in a tilted orientation the lowest point of the first flow loop moves through all points within the flow path of the first flow loop and this will generate gravity driven circulation of the liquid cell medium around the first flow loop wherein the tilted orientation places the movement axis at an angle in the range 5° to 85° away from the vertical.

The cell culture device may comprise a second flow loop comprising a second reservoir for holding a liquid cell medium and a second perfusion channel, wherein the second reservoir comprises a second reservoir first end and a second reservoir second end, and the second perfusion channel provides fluid communication between the second reservoir first end and the second reservoir second end thereby providing a looped flow path for a one-way gravity-driven flow of the liquid cell medium within the second flow loop when the cell culture device is tilted and moved by the movement system; wherein the cell culture device is configured such that when the cell culture device is moved with the movement axis in a tilted orientation the lowest point of the second flow loop moves through all points within the flow path of the second flow loop and this will generate gravity driven circulation of the liquid cell medium around the second flow loop. The second flow loop may be provided as a part of the same component as the first flow loop, e.g. on the same substrate, or there may be two separated components, e.g. on two substrate parts, that can be placed together to provide a device with two flow loops.

The one or more chambers may be separated from the second perfusion channel by a semipermeable barrier for selective transport of cell media and/or for selective growth or migration of living cells.

The first reservoir and/or the second reservoir may be formed in a U-shape so that the first and second ends of the respective reservoir are located at the beginning and the end of the U-shape respectively.

Thus the perfusion channel extends from the tips of the respective U-shaped reservoir in order to provide a path way to enclose the open end of the U-shaped reservoir and to complete a circuit involving the reservoir and the perfusion channel.

The one or more chambers may be provided between the first and second perfusion channels. Thus the chamber may be in contact with the first perfusion channel on one side of the chamber, and may be in contact with the second perfusion channel on the opposite side of the chamber.

The first reservoir may be smaller than the second reservoir and the first reservoir may be provided radially inwardly of the second reservoir. Thus the first flow loop may have a smaller perimeter than the second flow loop and the second flow loop may be provided so that its circumference encloses the circumference of the first flow loop.

The one or more chambers may be provided within the gap between the circumference of the first flow loop and the circumference of the second flow loop. Thus the chamber may be in contact with the first perfusion channel on one side of the chamber, and may be in contact with the second perfusion channel on the opposite side of the chamber.

The cell culture device of the third aspect may also or alternatively comprise any of the other optional features discussed above in relation to the first aspect.

Viewed from a fourth aspect, broader than the third aspect, the present invention provides a cell culture device as described above, separate to the cell culture apparatus and its movement system. Thus, an aspect of the present invention comprises a cell culture device for the cell culture apparatus described above, the cell culture device comprising:
a first flow loop comprising a first reservoir for holding a liquid cell medium and a first perfusion channel, wherein the first reservoir comprises a first reservoir first end and a first reservoir second end, and the first perfusion channel provides fluid communication between the first reservoir first and the first reservoir second end thereby providing a looped flow path for a one-way gravity-driven flow of the liquid cell medium within the first flow loop when the cell culture device is tilted and moved by the movement system, and
one or more chambers for culturing of living cells or living organoids; wherein
the one or more chambers are separated from the first perfusion channel by a semipermeable barrier for selective transport of cell media and/or for selective growth or migration of living cells;
wherein a movement axis is defined as an axis of the cell culture device that passes through the cell culture device;
and wherein the cell culture device is configured such that when the cell culture device is moved with the movement axis in a tilted orientation the lowest point of the first flow loop moves through all points within the flow path of the first flow loop and this will generate gravity driven circulation of the liquid cell medium around the first flow loop.

As discussed above this device with its single flow loop may be placed in use alongside another similar device, with that second device hence having a second flow loop that may work together with the first flow loop. Alternatively, the first flow loop may be used by itself in a system needing only a single perfusion channel. The cell culture device of this fourth aspect may also or alternatively include any of the other optional features discussed above in relation to the first aspect.

The present invention also extends to the use of the cell culture device for cell culture and hence, in another aspect, the invention provides a method comprising: using the cell culture device described in either of the above aspects, providing a suitable liquid cell medium in the flow loop, providing a suitable living cell and/or suitable living organoid in the chamber, tilting and moving the cell culture device to move the cell culture device with the movement axis in a tilted orientation in order to move the lowest point of the flow loop through all points within the flow path of the flow loop, and thereby generating gravity driven circulation of the liquid cell medium around the flow loop. Preferably the tilted orientation places the movement axis at an angle in the range 5° to 85° away from the vertical, or narrower ranges of angle as is described above as an optional feature of the first aspect.

The method may use a cell culture device that may also or alternatively include any of the optional features discussed above in relation to the first aspect. The method may use a cell culture apparatus as in the first aspect, with the movement system being used to provide the required tilting and moving.

It should be noted that as used herein the term “vertical” should be understood to be vertical with reference to the up and down direction of gravity, and horizontal to be the perpendicular to that vertical direction. A vertical axis is parallel to the vertical direction. References to the orientation or relatively orientation of the cell culture apparatus or parts thereof are references to positioning whilst in use.

Certain example embodiments of the invention will now be described by way of example only and with reference to the accompanying drawings in which:

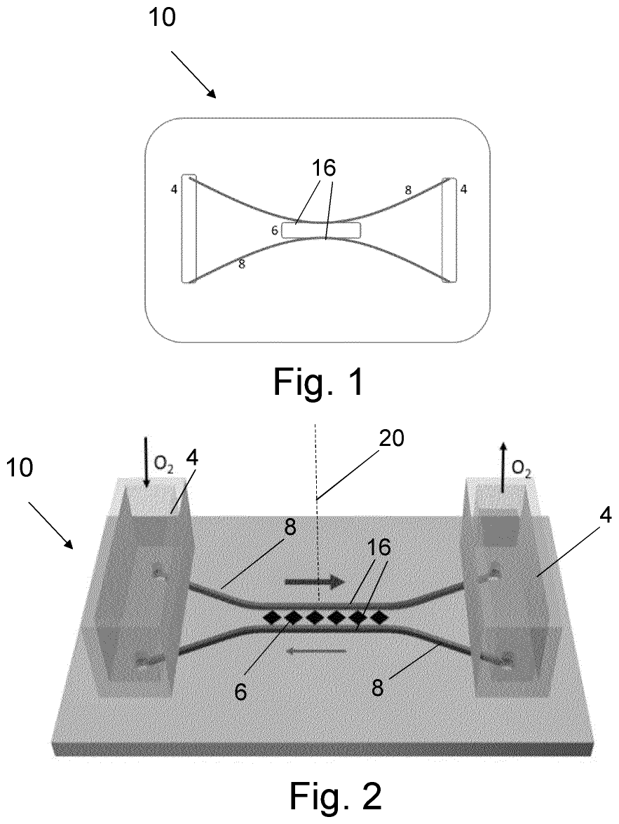

FIG. 1 is a schematic plan view of a proposed cell culture device;

FIG. 2 shows a similar cell culture device in perspective view;

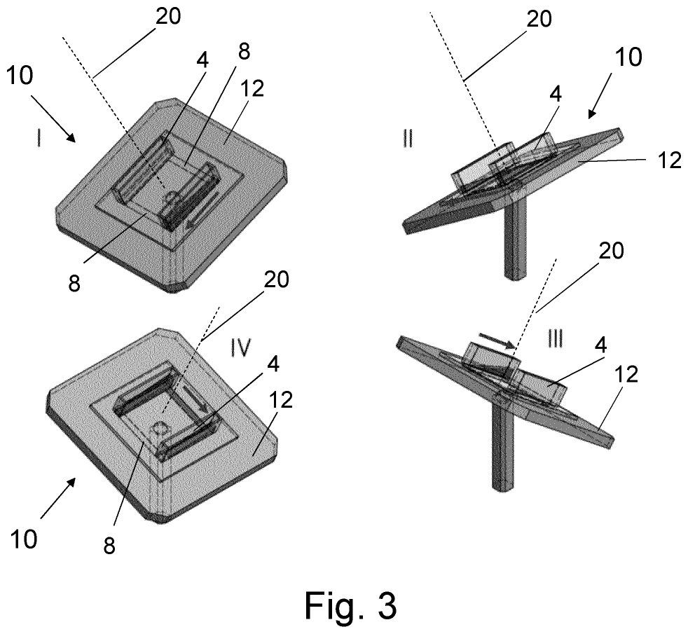

FIG. 3 illustrates a sequences of steps of a tilting movement for one-way gravity circulation of fluid in a cell culture device;

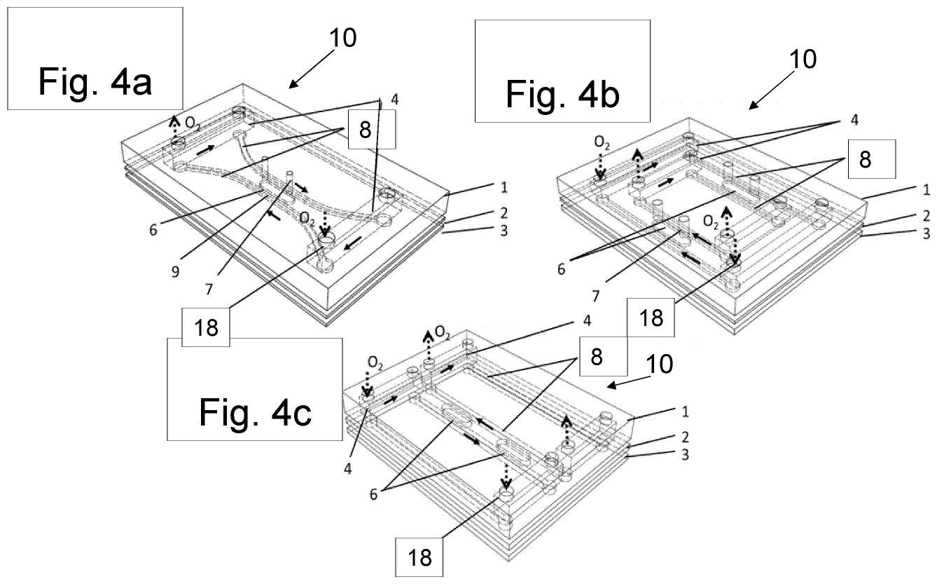

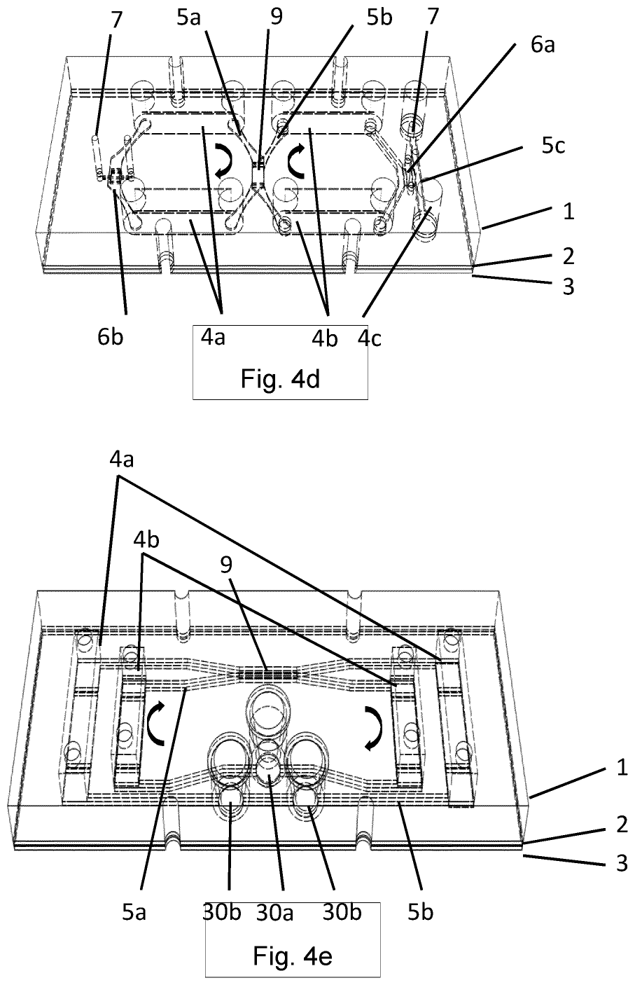

FIGS. 4a to 4e show varying designs for a fluid loop on the cell culture device;

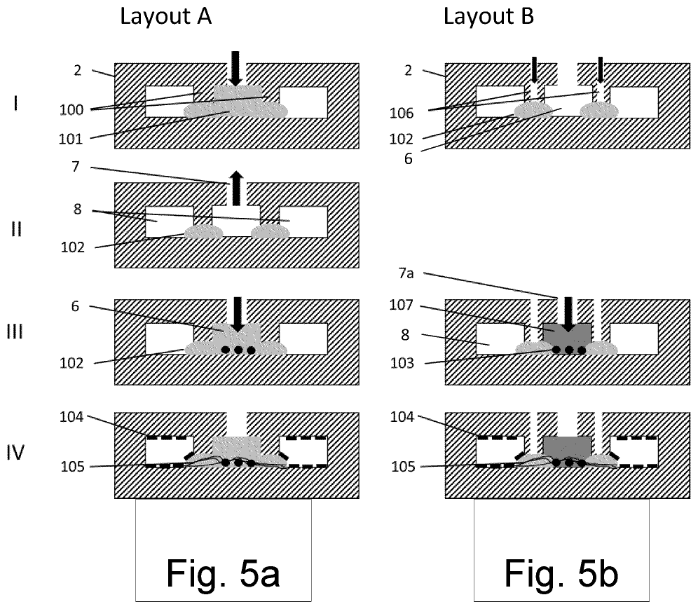

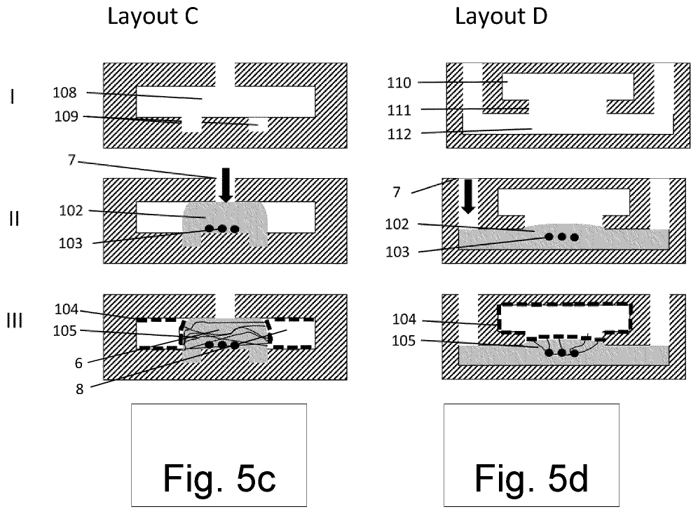

FIGS. 5a to 5d are schematic diagrams for four possible layouts for an extracellular matrix (ECM) barrier of a cell culturing chamber;

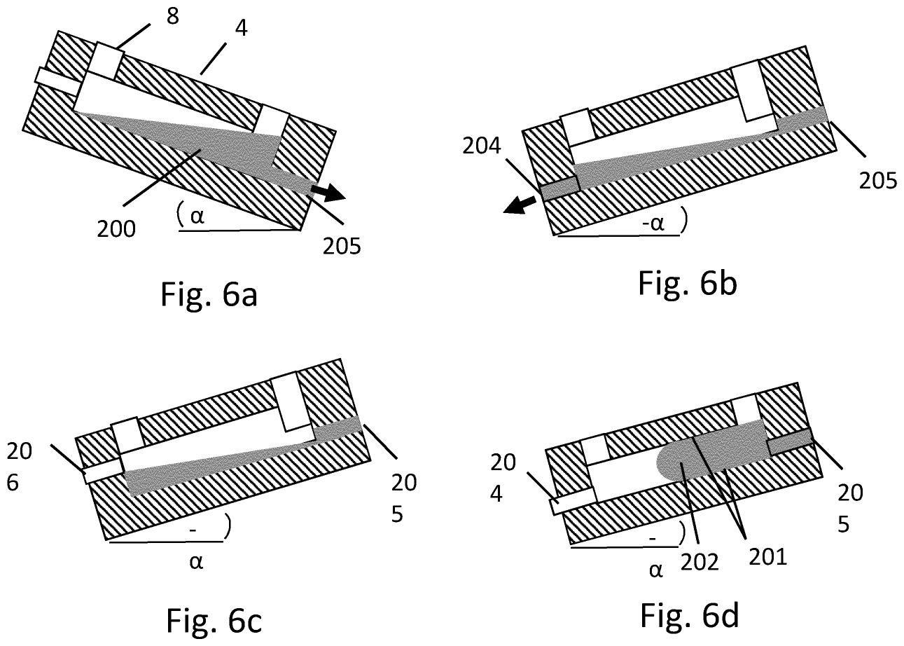

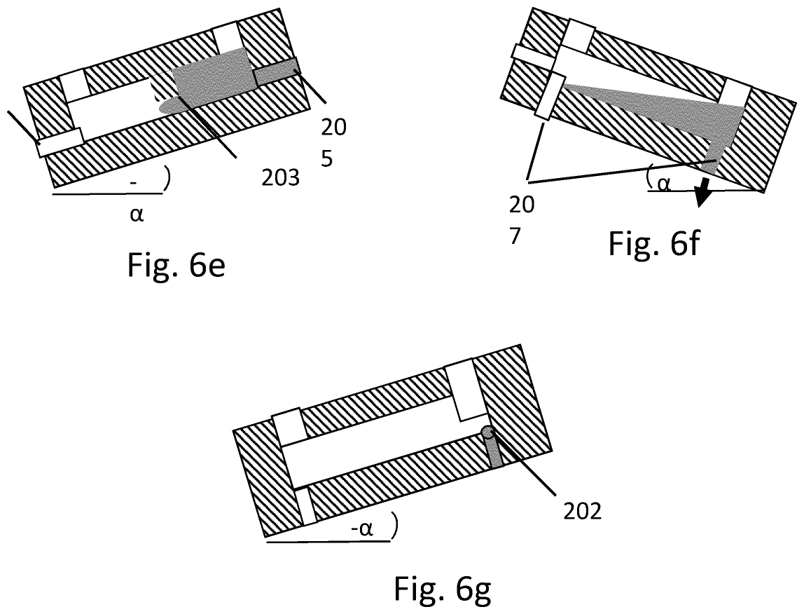

FIGS. 6a to 6g illustrate a simple flow path as well as various options for restricting reverse flow; and

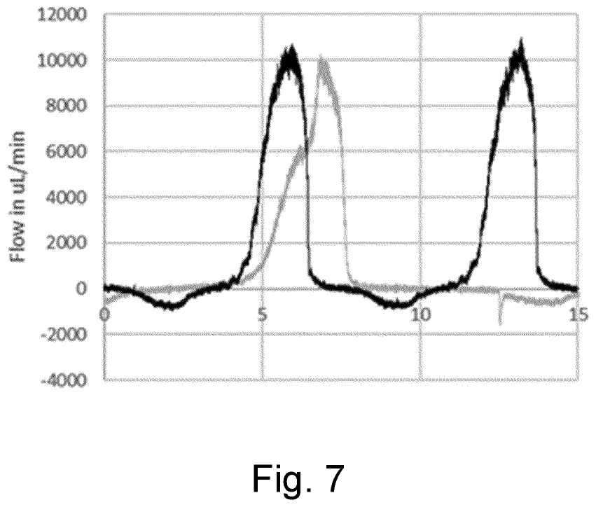

FIG. 7 shows a flow curve illustrating the flow rate achievable by an exemplary cell culture device.

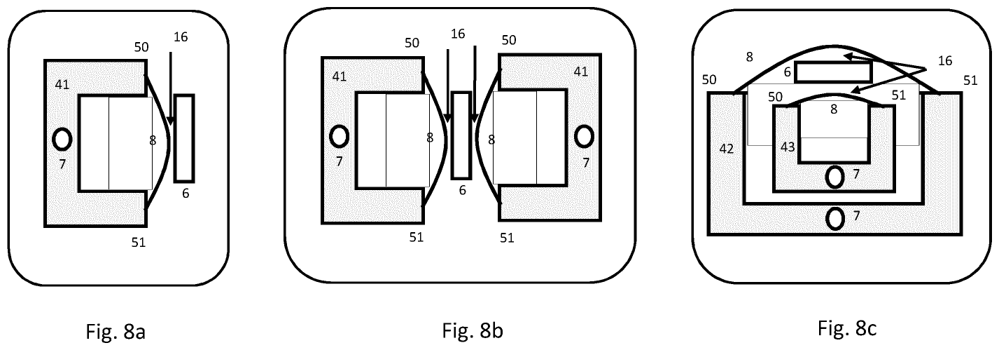

FIGS. 8a to 8c show alternative cell culture device configurations comprising one or two flow loops, each of the flow loops comprising one reservoir and one perfusion channel.

A cell culture device 10 is proposed herein, whereby a one-way gravity driven flow about a flow loop can be obtained via a tilting movement of the cell culture device 10. Example configurations for the cell culture device 10 are shown in FIGS. 1 and 2, with FIGS. 4a to 4c showing further variations. FIGS. 5a to 6d show additional detail of possible features for the cell culture device 10. The tilting movement may advantageously be provided by a cell culture system in which the cell culture device 10 is combined with a movement system 12, such as that shown schematically in FIG. 2. The movement system 12 can be a tilting platform of known type, with the cell culture device 10 hence being usable with typical laboratory equipment absent the need for additional investment in expensive devices. Alternatively, if appropriate, the cell culture device 10 might be provided for use with a bespoke movement system 12, that might be supplied separately.

As seen in FIGS. 1 and 2 the cell culture device has three main elements. Two reservoirs 4 are provided for holding and conveying a liquid cell medium. The reservoirs 4 provide fluid volumes as well as flow paths between two perfusion channels 8. The two reservoirs 4, together with the two perfusion channels 8, provide a flow loop for one-way gravity driven flow of the liquid cell medium. With a suitable tilting movement, as discussed further elsewhere herein, the liquid cell medium may flow from the first reservoir 4 into the first perfusion channel, through the first perfusion channel 8 to the second reservoir 4, through the second reservoir 4 and from the second reservoir 4 into the second perfusion channel, through the second perfusion channel 8 and then back to the second reservoir 4, before continuing on multiple circuits of that fluid loop. The perfusion channels 8 are separated from the chamber 6 via barriers 16, in this case on two sides of a single chamber 6, with the barriers 16 allowing for selective transport of cell media, such as transport of fluids, whilst blocking movement of other elements of the system, e.g. to retain cells or organoids within the chamber 6.

In the example of FIG. 3 the movement system 12, which may be a known laboratory tilting platform device as noted above, applies a tilting motion to the cell culture device 10 causing it to pass sequentially through positions I, II, III and IV. For example, the tilting platform device may raise and lower respective tilting actuators to cause the device to move between the four tilted positions. Optionally it may first move from a horizontal position to a first one of positions I, II, III and IV at the start of the use of the cell culture device 10. Similarly, optionally it may move to a horizontal position from a last one of positions I, II, III and IV at the end of the use of the cell culture device 10. This can be a movement of a constant speed or it may have a varying speed, including use of pauses, to provide a pulsatile flow pattern. The tilting movement causes the lowest point (i.e. lowest in height with reference to gravity) of the flow loop to move around the flow loop made of the reservoirs 4 and perfusion channels 8, and that in turn drives the one-way flow about the flow loop. The same one-way flow will occur with cell culture devices 10 having variations on the arrangement of the reservoirs and perfusion channels, for example as in FIGS. 1, 2 and 4a to 4c, since in each case there is a flow loop for fluid flow where the flow paths are in a circuit about a central point of the device, and hence the tilting movement will prompt one-way flow around the flow loop.

The movement of the cell culture device 10 by the movement system 12 can be envisaged with reference to a suitable movement axis, such as the axis 20 shown in FIGS. 2 and 3. This is an axis 20 normal to a plane of the cell culture device 10 and passing through it so that in this example the flow loop, i.e. the sequence of reservoirs 4 and channels 8, passes around the axis 20. With the tilting movement shown in FIG. 3 then the movement axis would have a rotating “stirring” motion wherein a reference point on the axis 20 (e.g. above the cell culture device 10) would trace around an outline of a square. Other movements could also be used, such as to tilt and rotate the movement axis 20 with a circular movement, and/or to rotate the cell culture device 10 about the movement axis 20 whilst the movement axis 20 is tilted.

It will be appreciated that the speed of the tilting movement and the maximum angle of the tilt can be controlled/adjusted as discussed elsewhere herein. That can allow for control/optimisation of performance such as in terms of the speed of flow, the rate of circulation, and/or the WSS within the flow paths.

As the flow is driven by gravity, liquid reservoirs 4 are needed that can fill and release the media volume during the tilting operation. Elongated (rectangular, elliptic, ring-shaped) reservoirs 4 on two sides of the flow loop are suitable because the liquid level is tilted in them as shown in FIG. 3, thus preventing backflow. The image also shows the flow direction (red) and the movement of the platform (black). Additional surface treatment e.g. via laser-engraving can be used to increase the wetting behaviour in the reservoir thus further preventing backflow. The enhanced wetting will keep remaining liquid in the emptied reservoir in place when the platform tilts back so that almost no media will flow back in the opposite direction. Placing the inlets and outlets in the open side channel/reservoirs 4 on different levels can also be used to reduce the amount of liquid flowing back during normal circulation. The flow rate depends on tilting angle, the channel dimensions, the width and the volume in the reservoirs and thus can be tuned by both, the designer and the end-user. Importantly, no pumps are needed to operate the device and several circuits can be run in parallel on one tilting platform allowing a robust scalability. Different organ models can be placed one after the other to study cross-organ toxicity and other effects.

By controlling tilting speed and angle we can precisely tune the flow and/or pressure in the channels without the need for external pumps. This allows to implement an atrial and a venous channel on one chip with different WSS/flow. An exemplary flow curve is given in FIG. 7. The flow was measured with a modified micro-Particle-Imaging-Velocimetry setup, and 19 degrees centigrade and for revolution rates of 4 rpm (shown in grey) and 8 rpm (shown in black) of the cell culture device. The flow rate is measured at a point within one of the perfusion channels so that during one revolution of the cell culture device one peak in the measured flow rate is seen. The curve demonstrates that fluid flows through the perfusion channel at a rate similar to physiological flow rates and that backflow is effectively prevented. Furthermore, an oxygen rich and an oxygen depleted side (red: arteria/blue: venous can be achieved by actively reducing/increasing the oxygen content in the reservoir, e.g. with a chemical reaction that consumes oxygen or by integrating a barrier (lung-on-chip model, see FIG. 2). Having arterial and venous components in a scalable OoC platform allows to develop far more complex cell, organoid or tissue structures than possible with current gravity based OoC platforms. Between both channels, cultivation chambers are placed that are later filled with endothelial cells and cells/organoids of interest either without or with hydrogel of different compositions to enable vascularization on-chip and building different OoC models depending on end-user needs. The present examples include a perfusion system with two separated circuits (mimicking an arterial and a venous channel) during tilting. The inventors have thus shown that several circuits can be run parallel, which underlines the scalability of the proposed device.

FIGS. 4a to 4e show further examples of the cell culture device 10. These devices 10 consists of a thick connection plate 1 with the reservoirs 4 having suitable fluidic connections 18 along with cell injection ports 7. The device can have a layered construction with one or more flow layers 2 integrating the perfusion channels 8 and the chamber 6, with a closing layer 3 at the bottom, i.e. at the opposite side to the connection plate 1. The chamber 6 is oriented in the middle of two perfusion channels 8 and separated from direct flow (straight arrow) by the barrier 16, which in this example comprises a layer of extracellular matrix (ECM) 9.

Different fluidic circuits are proposed:

A. As shown in FIG. 4a, one or more chambers 6 aligned between an arterial and venous channel 8. The arterial side is achieved by actively adding oxygen (dashed arrow) whereby a venous channel is generated by reducing the oxygen level.

B. As in FIG. 4b, two separated fluidic circuits, one in the middle and one outside, whereby one is forming an arterial and the other a venous circuit by adding or removing oxygen (dashed arrow). The cell cultivation chambers 6 are aligned between both circuits.

C. As in FIG. 4c, two separated fluidic circuits in close proximity to each other, one featuring a high oxygen level and flow rate (arterial side) and one with low oxygen level and flow (venous side). The cell cultivation chambers 6 are aligned between both channels.

D. The layout in FIG. 4d contains two separate circuits as proposed in FIG. 4c with the difference that no cell cultivation chambers, but a (semi)-permeable membrane 9 is added between both circuits thus adding a transport model (organ) to the chip. Different cells e.g. endothelial or epithelial cells can be seeded on one or both sides of the membrane 9 to allow an (active) exchange from one circuit to the other. Both perfusion channels 5a and 5b are placed in different layers and connected to separate reservoirs 4a and 4b and one or more cell cultivation chambers 6a and 6b on both sides. The cell chambers 6a and 6b can be shaped differently as described in FIGS. 5a to 5d and contain several cell loading ports 7. The layout in FIG. 4d can comprise a drainage channel 5c on one or both sides of the device. These can be connected to one or more drain reservoirs 4c.

E. The layout in FIG. 4e is similar to the configuration of FIG. 4d with the difference that the placement of the reservoirs 4a/b and perfusion channels 5a/b is similar to the one in FIG. 4b and a (semi)-permeable membrane 9 is placed between both circuits 5a/b. Due to the configuration of the reservoirs, the flow in both circuits is in phase and pointing in the same direction and not alternating and reversed like in FIG. 4d. Analog to FIG. 4d, different cells e.g. endothelial or epithelial cells can be seeded on one or both sides of the membrane 9 to allow an (active) exchange from one circuit to the other. The cell chambers can be configured like in FIG. 4d or as highlighted in the FIG. 5. In this particular configuration, a specific model is used. So-called Transwell® inserts 30 are introduced into the connection plate 1. Transwells® inserts are plastic containers sealed with a semi-permeable membrane at the bottom. Cells can be cultured on the membrane e.g. to generate a transport model 30a to study the uptake of a substance. This can be for instance a skin, lung or kidney model. Moreover, Transwells® inserts can include organoids or other 3d-models embedded in an ECM 30b to mimic metabolism or substance uptake of a target organ. Both, several Transwell® inserts-based models and specifically structured models as described in FIG. 5 can be mixed in one device.

Microfluidic cell culture devices 10 as in the current examples can be manufactured by laser structuring of thermoplastic sheets with varying thickness (currently Polymethyl-methacrylate (PMMA)). Besides, other thermoplastics like polycarbonate (PC), Cyclic-Olefin-Copolymers (COC), Polystyrene (PS) or Polyethylene-terephthalate (PET) are possible. The substrates are later UV activated and thermally bonded in a hot-press. Possible other manufacturing technologies include hot-embossing, injection moulding, micro milling or 3D printing. Moreover, the chip can be manufactured using the established soft-lithography process by casting the structures from a master mould in a silicone elastomer like PDMS and later bonding it to a glass slide.

One major functionality of the proposed cell culture device 10 is the ability to protect the cells from direct flow while allowing modelling of connection to blood vessels (vascularization), interaction with circulating cells such as (see above) and innervation. In contrast to other developments, we are not integrating an artificial barrier 16 but propose four different fluidic layouts (A, B, C, D) to structure a biodegradable ECM 9 between the cell chamber 6 and the perfusion channels 8. The forming of the ECM barrier 9 and loading of cells can be performed in three or four steps (I, II, III, and IV) as illustrated in FIG. 5a, FIG. 5b, FIG. 5c and FIG. 5d. FIG. 5a shows formation of a proposed “Layout A”, which integrates “bridges” with different heights 100 in the flow layer(s) 2 between the perfusion channels 8 and the cell chamber 6. In a first step (I), a liquid hydrogel 101 is injected via the loading port 7 at 4 degrees and transported via capillary forces into the “bridges”. Next (II), the liquid hydrogel is removed with a slight vacuum so that Hydrogel only remains within the bridge 102. The hydrogel sticks due to capillary forces and forms a meniscus. After polymerization, cells or organoids or tissues 103 are loaded through the injection port 7 and the chamber 6 is filled with hydrogel (III). Finally, endothelial cells 104 are loaded on both sides of the perfusion channels 8 and form a vascular network 105 connecting the organoids with the perfusion channels (IV).

Besides, it is possible to load the hydrogel (101) and the organoids, cells (103) in one step (refer to step III) if only one type of cells is used.

Another chamber layout “Layout B” is shown in FIG. 5b. Layout B also integrates “bridges” with different heights 100 in the flow layer(s) 2 between the perfusion channels 8 and the cell chamber 6. Layout B comprises additional channels 106 within the bridges 102 to allow for the injection of a hydrogel in step I. Due to capillary forces, the hydrogel remains in the small gaps beneath the bridges 102. This allows a similar or different cell/hydrogel composition 107 to be loaded into the middle cell culture chamber 6 in step III. Similar to Layout A, endothelial cells (104) can be seeded to form micro capillaries (105) in step IV.

Turning to FIG. 5c, in contrast to Layout A, a proposed “Layout C” uses small ridges or dead channels 109 placed underneath the place where perfusion channels 8 and cell cultivation chamber 6 should be separated. All channels have here the same height and form a big chamber 108. After cleaning and drying of the device (in step I) a liquid hydrogel 102 containing cells or organoids 103 is injected via the injection port 7 (in step II) and the hydrogel is polymerized. The amount of ECM injected is controlled in that way so that the hydrogel fills the small ridges first thus forming a meniscus separating the cell chamber 6 and the perfusion channels 8. The cells will then be immobilized in the hydrogel and analog to layout endothelial cells 104 are flushed in on both perfusion channels 8 and finally form a vascular network 105 (in step III).

Another “Layout D” features a cell separation layer in a multilayer setup with the perfusion channel(s) 110 on-top of the cell culture chamber 112. This is shown in FIG. 5d. The perfusion channel 110 and the cell culture chamber 112 are horizontally separated by a window 111, the window being narrower than the perfusion channel 8 and cell culture chamber 112 as shown. After cleaning and drying of the device (in step I) a liquid hydrogel 102 containing cells or organoids 103 is injected via the injection port 7 (in step II) and the hydrogel is polymerized. The cell culture chamber 112 is shaped as a channel beneath the perfusion chamber 110 and connected to the surface so that the cells/organoids can be placed underneath the perfusion channel by pipetting. The cells will then be immobilized in the hydrogel and analog to Layout A endothelial cells 104 can be flushed in on the perfusion channel and finally form a vascular network 105 (in step III).

We furthermore propose certain layouts and/or other features for the liquid reservoirs 4 and/or perfusion channels in order to obtain a directed flow and/or to inhibit reverse flow, as shown in FIGS. 6a to 6g. When the reservoirs 4 are tilted by an angle of a, the liquid 200 in the reservoir 4 is tilted by this angle too because it is opened to the atmosphere at the filling ports 8. In FIG. 6a this leads to the effect that the liquid leaves the outlet 205 in the desirable flow direction. Nevertheless, remaining liquid in the reservoir might cause an unwanted backflow through the inlet 204 when the reservoir is tilted in the opposite direction (−α) as shown in FIG. 6b. We propose four different layout improvements to reduce the backflow due to this effect:

A. As shown in FIG. 6c the inlet 206 is created within the reservoir layout 4 at a higher level than the outlet 205, thus preventing backflow when it is tilted in the opposite direction (−α).

B. An alternative or additional feature, as shown in FIG. 6d, is laser engraving of the reservoir surface 201 to decrease wetting in order that the contact angle between the liquid and the surface is increased, resulting in the promotion of a meniscus that will prevent remaining liquid to be tilted back into the inlet 204.

C. Another possibility, as in FIG. 6e, is to reduce the height of the reservoir 4 so that the meniscus can be further increased to form a so-called capillary stop valve 203 that prevents backflow.

D. An alternative reservoir design is shown in FIG. 6f and FIG. 6g. Here, the liquid enters and leaves the reservoir through holes 207 from underneath. This alternative inlet/outlet configuration will result in the formation of a capillary-stop valve at the outlet at which a meniscus 202 will be formed at the air-liquid interface which prevents inflow of liquid into the chamber through the outlet.

All four layouts for the liquid reservoirs 4 and/or perfusion channels/methods for obtain a directed flow and/or to inhibit reverse flow can be combined to promote a fully one-directional flow.

Another alternative layout for providing the cell culture device is proposed in FIG. 8a. The layout comprises one flow loop which comprises a single reservoir 41. The single reservoir comprises a first end 50 and a second end 51. A perfusion channel 8 connects the first end 50 of the reservoir to the second end 51 of the reservoir. A flow loop is therefore provided via a single reservoir 41 and a single perfusion channel. The perfusion channel 8 is in contact with a cell culture chamber 6 separated by a barrier 16. Directionality of the flow within the flow loop is achieved by the use of a specifically-shaped reservoir. In the example shown a U-shaped reservoir 41 is provided. As the U-shaped reservoir is tilted as shown in FIG. 6, the 3D tilting motion and the geometric confinement of the fluid in the reservoir forces the fluid to flow in a single direction as dictated by the tilting motion, thus a directed flow is generated. One or more venting holes/filling ports 7 are included in the reservoir 41 to allow a defined filling of the reservoir and to remove the air in the system.

FIG. 8b shows a further alternative layout for the cell culture device. The layout is an adaptation of that shown in FIGS. 1 and 2 in which two of the single reservoir flow loops as shown in FIG. 8a are provided in order to allow the cell culture chamber to contact two perfusion channels. The device shown comprises two separate flow loops so that the cell culture chamber is contacted by two perfusion channels each belonging to a separate flow loop.

FIG. 8c shows a further alternative layout for the cell culture device. In this example, two separate flow loops each comprise a single reservoir 42, 43 and a single perfusion channel 8. The second flow loop is disposed radially outwardly of a first flow loop. The layout is an adaptation of that shown in FIG. 4b in which one flow loop is provided in the middle and one outside with a cell culture chamber disposed between the respective perfusion channels. In the example shown, the second flow loop comprises a bigger U-shaped reservoir 42 which is placed around a smaller U-shaped reservoir 43 belonging to the first flow loop. Both reservoirs 42, 43 may comprise one or more venting hole/filling port 7. A cell culture chamber 6 is placed between perfusion channel of the first flow loop and the perfusion channel of the second flow loop. Each perfusion channel 8 is separated from the cell culture chamber 6 by a barrier 16 as described above.

Analog to the layout with two reservoirs and two connecting channels, the previously mentioned methods to improve directionality of the flow, discussed with reference to FIGS. 6a to 6g, may be applied to the layout shown in FIGS. 8a and 8b also.

For scalable fabrication of the cell culture devices it is proposed to use CO₂ laser cutting/etching, with subsequent UV activation and thermal bonding of Poly(methyl methacrylate) (PMMA) sheets. PMMA is a polymer widely used in cell cultivation due to its good optical properties, its biocompatibility and due to the fact that it can be easily machined with CO₂ lasers. In contrast to the elastomer Polydimethylsiloxane (PDMS) that is widely used in microfluidics, PMMA has a low adsorption of small molecules and is impermeable for oxygen which makes it suitable for separating arterial and venous flow and hence for creating a more physiological circulation than currently standard. Moreover, PMMA can be processed like other thermoplastics using hot-embossing or injection moulding and is therefore better suited for mass production than PDMS. The inventors have established that endothelial cells (HUVECs) can be cultivated in the device and invade liver organoids that are embedded in an extracellular matrix (ECM).

For a proper functionality of the HUVECs (alignment, sprouting) a sufficiently high WSS (>1 dyne/cm2) and a uni-directional flow are needed. The inventors have found that, with the proposed cell culture device 10, this can advantageously be achieved using a standard tilting platform as the movement system 12 (for example, a Mimetas OrganoFlow® rocker, as provided by MIMETAS B.V., of Leiden, The Netherlands).

The proposed cell culture device 10, which is moveable on such a tilting platform to achieve one-way flow, thus has the following advantages over other solutions on the market:
Flow directionality.
Precisely controllable flow rate and pressure in the system.
Easy to handle scalable platform that fits with standard laboratory processes.
Only a low amount of cell cultivation media is needed.
Experiments can be parallelized which makes it suitable for drug screening.
Several tissues/organoids can be combined in one circuit.
Platform can be adapted to circulating blood, cancer and immune cells, bacteria and virus particles.
Different flow rates and oxygen levels can be integrated in one device without using pumps.
Only a standard tilting support device is needed (no additional laboratory equipment).
Setup fits in standard incubators without tubes (no risk of contamination).
Air bubbles are automatically trapped in the reservoirs and do not enter the channel.

We claim:

1. A cell culture apparatus comprising: a cell culture device; and a movement system for moving the cell culture device;
the cell culture device comprising: at least two reservoirs for holding a liquid cell medium, one or more chambers for culturing of living cells, tissues or living organoids and at least two perfusion channels connecting the reservoirs; wherein the one or more chambers are separated from at least one of the perfusion channels by a semipermeable barrier for selective transport of cell media and/or for selective growth or migration of living cells; and wherein the reservoirs and perfusion channels are in fluid communication with one another in order to form a flow loop having a flow path for a one-way gravity-driven flow of the liquid cell medium when the cell culture device is tilted and moved by the movement system;
a movement axis being defined as an axis of the cell culture device that passes through the cell culture device with the flow loop being located around the movement axis; and
the movement system being configured to move the cell culture device with the movement axis in a tilted orientation in order to move the lowest point of the flow loop through all points within the flow path of the flow loop and thereby generate gravity driven circulation of the liquid cell medium around the flow loop, wherein the tilted orientation places the movement axis at an angle in the range 5° to 85° away from the vertical.

2. A cell culture apparatus as claimed in claim 1, wherein the movement in a tilted orientation is a movement of the device whilst the axis is at an angle of 20° to 80° away from the vertical.

3. A cell culture apparatus as claimed in claim 1, wherein the barrier acts as a semi-permeable and/or selectively permeable barrier between the perfusion channel and the chamber, allowing for passage of certain cell media to provide a way of connecting the liquid cell medium to the at least one chamber.

4. A cell culture apparatus as claimed in claim 1, wherein the barrier is an extra cellular matrix (ECM) barrier, and/or wherein the barrier includes a biomaterial or extracellular natural material and comprises one or more of Matrigel®, Geltrex®, Collagen, a synthetic hydrogel, and/or a polymer membrane.

5. (canceled)

6. A cell culture apparatus as claimed in claim 1, comprising two barriers at two locations about the chamber, wherein the two barriers each provide for transport of certain cell media between the chamber and the two perfusion channels.

7. A cell culture apparatus as claimed in claim 1, wherein a part of the flow loop is configured to allow contact of the liquid cell medium with a gas from outside of the flow loop.

8. A cell culture apparatus as claimed in claim 1, the movement axis for the cell culture device being defined as an axis normal to a plane of the device, wherein during movement of the device by the movement system the movement axis is tilted in the sense that it is angled to the vertical thereby positioning lifting one section of the flow loop higher than another section of the flow loop, and wherein the position of the tilted axis moves in order to change the section(s) of the flow loop that are higher and lower relative to one another.

9. A cell culture apparatus as claimed in claim 1, wherein the tilted movement axis is moved by the movement system in a rotational fashion so that a reference point on the movement axis traces around the circumference of a two dimensional shape.

10. A cell culture apparatus as claimed in claim 1, wherein the movement in a tilted orientation includes a rotation of the cell culture device about the movement axis, such that a reference point on the flow loop moves in a circle about the tilted movement axis.

11. A cell culture apparatus as claimed in claim 1, wherein the flow path includes a flow reversal restriction feature comprising one or more of: a change in depth; a capillary stop valve; and/or a surface treatment promoting decreased wetting.

12. A cell culture apparatus as claimed in claim 1, wherein the flow loop comprises a looped flow path that passes, in sequence, from a first reservoir, then through a first perfusion channel and past a first barrier at a first side of the chamber, then to a second reservoir, then from the second reservoir, through a second perfusion channel and past a second barrier at a second side of the chamber, then returning to the first reservoir.

13. A cell culture apparatus as claimed in claim 1, wherein the reservoirs each comprise a volume of 40 to 900 μL for receiving liquid cell medium.

14. A cell culture apparatus as claimed in claim 1, wherein the perfusion channels each extend from the outlet of one reservoir to the inlet of another reservoir, passing by at least one chamber with a barrier providing a link to the chamber at a mid-portion of the channel; and wherein the length of each perfusion channel is in the range 10 to 30 mm.

15. A cell culture apparatus as claimed in claim 1, wherein the chamber comprises a volume or 2.5 to 120 μL for receiving the living cells or living organoids and the chamber is protected from shear force of the perfusion channels via the barrier(s).

16. A cell culture apparatus as claimed in claim 1, wherein the cell culture device includes the liquid cell medium therein and/or the chamber includes therein a living cell or living organoid.

17. A cell culture apparatus as claimed in claim 16, wherein the cell culture device includes a volume of 50 to 600 μL of the liquid cell medium.

18. A cell culture apparatus as claimed in claim 1, wherein the dimensions of the cell culture device as well as the tilting speed and angle of movement by the movement system are configured to provide flow rates within the range of physiological flow rates such that the apparatus is configured to provide a flow rate of 15 to 80 μL/s of the liquid cell medium in the flow path.

19. (canceled)

20. A cell culture apparatus as claimed in claim 1, wherein the dimensions of the cell culture device as well as the tilting speed and angle of movement by the movement system are configured to provide shear stresses within the range of physiological shear stresses such that the apparatus is configured to provide a shear stress in the range of 0.1 to 100 dyne/cm² for flow of the liquid cell medium in the flow path.

21. (canceled)

22. A cell culture device for the cell culture apparatus of claim 1, the cell culture device comprising:
at least two reservoirs for holding a liquid cell medium, one or more chambers for culturing of living cells or living organoids and at least two perfusion channels connecting the reservoirs;
wherein the one or more chambers are separated from at least one of the perfusion channels by a semipermeable barrier for selective transport of cell media and/or for selective growth or migration of living cells; and
wherein the reservoirs and perfusion channels are in fluid communication with one another in order to form a flow loop having a flow path for a one-way gravity-driven flow of the liquid cell medium when the cell culture device is tilted and moved by the movement system;
wherein a movement axis is defined as an axis of the cell culture device that passes through the cell culture device with the flow loop being located around the movement axis;
and wherein the cell culture device is configured such that when it is moved with the movement axis in a tilted orientation in order to move the lowest point of the flow loop through all points within the flow path of the flow loop then this will generate gravity driven circulation of the liquid cell medium around the flow loop.

23. A method for cell culture, the method comprising: using the cell culture apparatus as claimed in claim 1, providing a suitable liquid cell medium, cells and/or other components in the flow loop, providing a suitable living cell and/or suitable living cells/organoids/tissue in the chamber, tilting and moving the cell culture device to move the cell culture device with the movement axis in a tilted orientation in order to move the lowest point of the flow loop through all points within the flow path of the flow loop, and thereby generating gravity driven circulation of the liquid cell medium around the flow loop.
